(19) United States
(12) Patent Application Publication
(10) Pub. No.: US 2017/0273637 A1
(43) Pub. Date: Sep. 28, 2017

(54) INFORMATION PROCESSING DEVICE AND INFORMATION PROCESSING METHOD

(71) Applicant: SEIKO EPSON CORPORATION, Tokyo (JP)

(72) Inventor: Tatsuhiko SUGIYAMA, Matsumoto-shi (JP)

(73) Assignee: SEIKO EPSON CORPORATION, Tokyo (JP)

(21) Appl. No.: 15/453,396

(22) Filed: Mar. 8, 2017

(30) Foreign Application Priority Data
Mar. 23, 2016 (JP) 2016-058029

(51) Int. Cl. A61B 5/00; A61B 5/11

(52) U.S. Cl. CPC A61B 5/7278; A61B 5/11; A61B 5/742; A61B 5/6801

(57) ABSTRACT

An information processing device includes: an information acquisition unit that acquires biometric information from a biometric sensor; a processing unit that generates first report information for reporting stamina information indicating stamina of a user and second report information different from the first report information based on the biometric information; and an output unit that outputs the first report information and the second report information. The second report information includes at least one piece of information among second stamina information which indicates the stamina of the user and is different from the stamina information, third stamina information regarding another user, advice information or alert information regarding an activity of the user, and exercise continuable information regarding the user.

### BACKGROUND

1. Technical Field

The present invention relates to an information processing device and an information processing method.

2. Related Art

For example, in general, in a case in which walking such as mountain-climbing or hiking in which physical strength is consumed is continuously performed, it is important to make plans according to the physical strengths of activity performers. Therefore, in a case in which mountain-climbing or hiking is performed, it is general to prepare materials such as maps or guide books of destinations in advance and generate activity planes with reference to the materials. At that time, action schedules including distances or walking times up to the destinations, rest places, rest times, and the like are generated and actions are performed in accordance with the action schedules.

However, weather in mountains is easily changed and it is also necessary to correct an action in accordance with changes in environments. In a case in which it is necessary to determine whether to continue to climb or climb down a mountain in accordance with the changes in the weather in the mountain, it is necessary to consider conditions of climbers. However, the action schedules generated based on the materials such as maps or guide books are taken into consideration from the physical strengths of general people and do not match physical strengths of actual climbers in many cases. In cases of group actions, it can be said to be difficult to climb down alone or desire to continue climbing, and thus respective conditions may not be objectively ascertained.

On the other hand, in an information processing device disclosed in, for example, JP-A-2013-220182, the physical strength, a remaining activable time, a movable distance, or the like of a user is calculated using biometric information, environmental information, or the like of the user (for example, a climber). Then, this information is displayed on a display unit and is reported to the user. Thus, the user can select a future action with reference to the information displayed on the display unit.

It is desirable that information indicating the physical strength (stamina) of a user (hereinafter referred to as stamina information) is specified from biometric information or the like regarding a user (subject) and a user ascertains reported information more easily in a case in which the specified stamina information or the like is reported.

However, JP-A-2013-220182 discloses that the physical strength of a user or a remaining activable time, or the like is displayed on the display unit, but does not disclose how the information is reported to the user so that the user can ascertain the information more easily.

### SUMMARY

An advantage of some aspects of the invention is to provide an information processing device and an information processing method capable of easily outputting stamina information regarding a user so that the user can easily ascertain the stamina information.

An aspect of the invention relates to an information processing device including: an information acquisition unit that acquires biometric information from a biometric sensor; a processing unit that generates first report information for reporting stamina information indicating stamina of a user and second report information different from the first report information based on the biometric information; and an output unit that outputs the first report information and the second report information. The second report information includes at least one piece of information among second stamina information which indicates the stamina of the user and is different from the stamina information, third stamina information regarding another user, advice information or alert information regarding an activity of the user, and exercise continuable information regarding the user.

According to an aspect of the invention, the first report information regarding the stamina information regarding the user is generated, and at least one piece of report information is generated as the second report information among second stamina information which indicates the stamina of the user and is different from the stamina information, third stamina information regarding another user, advice information or alert information regarding an activity of the user, and exercise continuable information regarding the user, and this information is output. Thus, the user can confirm not only the stamina information regarding the user but also the second report information. Accordingly, it is possible to output the stamina information regarding the user so that the user can easily ascertain the stamina information.

According to the aspect of the invention, in a case in which the information acquisition unit is not able to acquire the biometric information from the biometric sensor, the processing unit may perform an estimation process of estimating the biometric information and estimate the stamina information based on coefficient information used for the estimation process.

Thus, even in a case in which the biometric information may not be acquired from the biometric sensor, it is possible to report the stamina information regarding the user to the user.

In the aspect of the invention, the processing unit may perform an estimation process of estimating the biometric information based on an activity time corresponding to an activity strength of the user and coefficient information corresponding to the activity time and estimate the stamina information based on the coefficient information used for the estimation process.

With this configuration, it is possible to reflect the activity time and the activity strength of the user in the stamina information.

In the aspect of the invention, the information acquisition unit may acquire post-activity biometric information from the biometric sensor after the activity of the user stops. The processing unit may estimate the stamina information based on the post-activity biometric information.

With this configuration, it is possible to add the post-activity biometric information after the activity of the user is stopped and estimate the stamina information.

In the aspect of the invention, the processing unit may estimate the stamina information based on a change in the biometric information by an activity continued by the user.

With this configuration, it is possible to add the change in the biometric information in a case in which the activity continued by the user is performed and estimate the stamina information.

In the aspect of the invention, the processing unit may generate stamina information in an uphill exercise or stamina information in a downhill exercise as the second stamina information.

With this configuration, the user can ascertain the stamina information in the uphill exercise or the downhill exercise.

In the aspect of the invention, the processing unit may generate information regarding an animation in which a character is used, as the first report information.

With this configuration, the user can confirm the animation displayed on the display unit and can ascertain an overview of the own stamina information at first glance.

In the aspect of the invention, the processing unit may receive stamina correction information for correcting the stamina information from the user and correct the stamina information based on the stamina correction information.

With this configuration, it is possible to display the report information of the stamina information closer to the stamina assumed subjectively by the user.

Another aspect of the invention relates to an information processing method including: acquiring biometric information from a biometric sensor; generating report information for reporting stamina information indicating stamina of a user as first report information based on the biometric information; generating at least one piece of report information among second stamina information which indicates the stamina of the user and is different from the stamina information, third stamina information regarding another user, advice information or alert information regarding an activity of the user, and exercise continuable information regarding the user, as the second report information; and outputting the first report information and the second report information.

Still another aspect of the invention relates to a program causing a computer to function as each of the foregoing units.

### BRIEF DESCRIPTION OF THE DRAWINGS

The invention will be described with reference to the accompanying drawings, wherein like numbers reference like elements.

FIG. 1 is a diagram illustrating an example of a system configuration according to an embodiment.

FIG. 2 is a diagram illustrating a display example of report information related to stamina information.

FIG. 3 is a diagram illustrating another display example of the report information related to stamina information.

FIG. 4 is a diagram illustrating still another display example of the report information related to stamina information.

FIG. 5 is a diagram illustrating still another display example of the report information related to stamina information.

FIG. 6 is a diagram illustrating a display example of second report information related to second stamina information.

FIG. 7 is a diagram illustrating a display example of the second report information related to third stamina information.

FIG. 8 is a diagram illustrating a display example of the second report information related to advice information.

FIG. 9 is a diagram illustrating a display example of the second report information related to alert information.

FIG. 10 is a diagram illustrating a display example of the second report information related to exercise continuable information.

FIG. 11A is a diagram illustrating a display example of report information of stamina information using an animation.

FIG. 11B is a diagram illustrating another display example of report information of stamina information using an animation.

FIG. 11C is a diagram illustrating still another display example of report information of stamina information using an animation.

FIG. 11D is a diagram illustrating still another display example of report information of stamina information using an animation.

FIG. 11E is a diagram illustrating still another display example of report information of stamina information using an animation.

FIG. 12 is a flowchart illustrating the flow of a process according to the embodiment.

FIG. 13 is a diagram illustrating a stamina information estimation process.

FIG. 14 is a diagram illustrating an activity strength and an activity time.

FIG. 15 is a diagram illustrating a heartbeat drift phenomenon.

FIG. 16 is a flowchart illustrating the flow of a process according to a modification example.

FIG. 17 is a diagram illustrating a relation between a pace and an altitude at the time of mountain-climbing.

FIG. 18 is a diagram illustrating a heart rate at the time of mountain-climbing.

FIG. 19 is a diagram illustrating stamina correction information.

FIG. 20 is a diagram illustrating the outer appearance of an electronic apparatus.

FIG. 21 is another diagram illustrating the outer appearance of the electronic apparatus.

FIG. 22 is still another diagram illustrating the outer appearance of the electronic apparatus.

FIG. 23 is a diagram illustrating an information processing system.

FIG. 24 is another diagram illustrating an information processing system.

FIG. 25 is still another diagram illustrating an information processing system.

### DESCRIPTION OF EXEMPLARY EMBODIMENTS

Hereinafter, an embodiment will be described. The embodiment to be described below does not appropriately limit content of the invention described in the appended claims. All of the configurations to be described in the embodiment are not requisite constituents of the invention.

### 1. Example of System Configuration

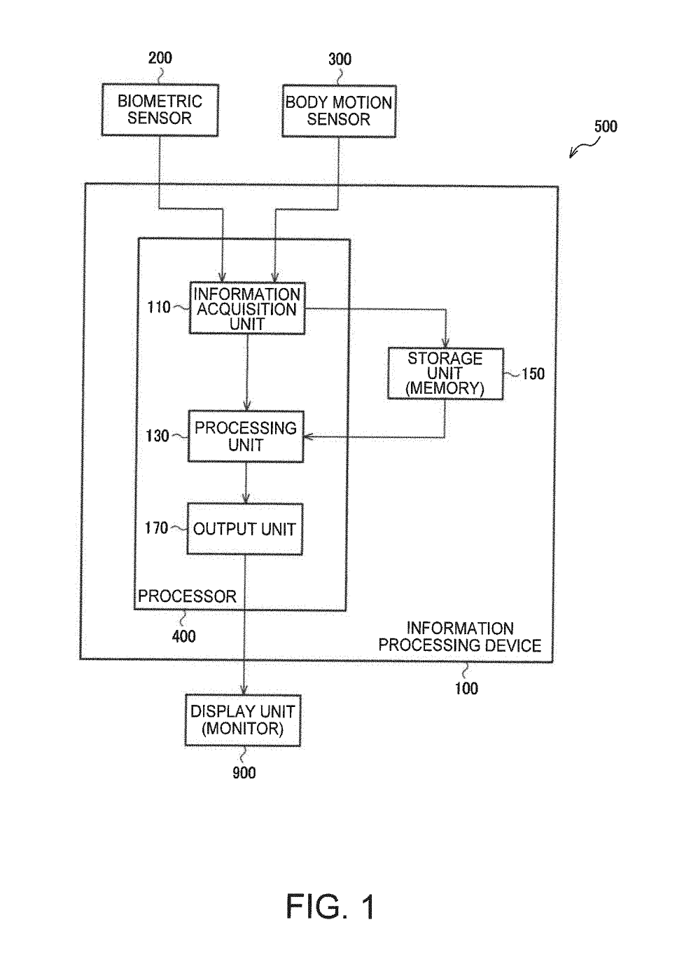

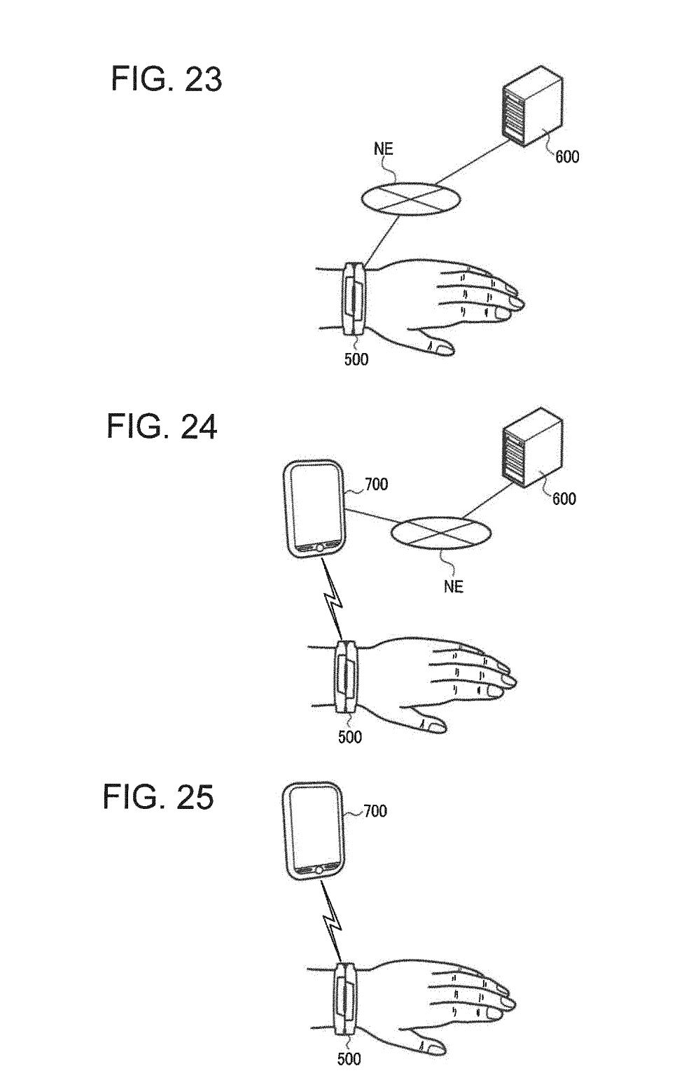

FIG. 1 illustrates a configuration example of an information processing device 100 (biometric information processing device or an information processing system) and an electronic apparatus 500 including the information processing device 100 according to the embodiment. The information processing device 100 according to the embodiment includes an information acquisition unit 110, a processing unit 130, a storage unit (memory) 150, and an output unit 170. As an example of the electronic apparatus 500 including the information processing device 100, a wearable apparatus including a biometric sensor 200, a body motion sensor 300, a processor 400, and a memory 150 can be exemplified. The electronic apparatus 500 may include a display unit (monitor) 900 or the like. Some or all of the functions of the information processing device 100 according to the embodiment are realized by, for example, a wearable apparatus. Here, some or all of the functions of the information processing device 100 (information processing system) may be realized by, for example, an electronic apparatus (portable electronic apparatus) different from a wearable apparatus such as a smartphone or a server system. A specific configuration example of the information processing system will be described below with reference to FIGS. 23 to 25. The information processing device 100 and the electronic apparatus 500 including the information processing device 100 are not limited to the configuration in FIG. 1. Various modifications can be made by omitting some of the constituent elements or adding other constituent elements.

Next, a process performed by each unit will be described. The information acquisition unit 110 acquires biometric information regarding a subject from the biometric sensor 200. The information acquisition unit 110 can also acquire body motion information regarding the subject from the body motion sensor 300. The biometric information is detected by, for example, the biometric sensor 200 installed in the wearable apparatus 500 worn by the subject. Similarly, the body motion information is detected by, for example, the body motion sensor 300 installed in the wearable apparatus 500.

For example, as will be described below with reference to FIG. 23, in a case in which the information processing device 100 according to the embodiment is realized in a server system 600 and the server system 600 acquires biometric information and body motion information from the wearable apparatus 500 worn by a user, the information acquisition unit 110 may be a communication unit (a reception unit that receives information from the wearable apparatus 500) that communicates with the wearable apparatus 500 via a network. The communication unit may be, for example, a communication device such as a USB connector (communication terminal) or a wireless antenna or may be a processor or the like controlling a communication device.

The biometric sensor 200 (biometric sensor device) is a sensor that is installed in the wearable apparatus 500 worn by the subject and can acquire biometric information regarding the subject. For example, the biometric information is information indicating a biometric activity of the subject and acquired from the biometric sensor 200. The biometric information is, for example, information indicating a heart rate (pulse rate) or body temperature, a blood pressure, a blood flow, an activity time (sleep time), or an activity state (exercise state). For example, in a case in which the information acquisition unit 110 acquires pulse wave information as biometric information, a pulse wave sensor is employed as the biometric sensor 200. The pulse wave information is information regarding a pulse wave of a subject and is, for example, the above-described heart rate (pulse rate). The pulse wave sensor is a sensor that detects pulse wave information (a pulse wave signal). For example, a photoelectric sensor or the like including a light-emitting unit and a light-receiving unit is considered. It is known that the pulse wave sensor can be realized by any of various sensors such as a photoelectric sensor and another type of sensor (for example, an ultrasonic sensor). Such a sensor can be broadly applied as the pulse wave sensor according to the embodiment. The biometric sensor 200 may be configured to include a blood pressure sensor, a temperature sensor, or the like.

The body motion sensor 300 (body motion sensor device) is a sensor that is installed in the wearable apparatus 500 worn by the subject and can acquire body motion information regarding the subject. For example, the body motion information is information indicating a body motion of the subject and acquired from the body motion sensor 300. The body motion information is, for example, information indicating a movement distance of the subject, the number of steps, a step length, a movement time, a movement speed, acceleration, an absolute amount of a variation in acceleration, a frequency of a variation in acceleration, an exercise amount, exercise content (activity content), an altitude difference per certain unit time, an altitude, information acquired from a gyro sensor, an absolute amount of a variation in an angular velocity, a frequency of a variation in an angular velocity, information acquired from a geomagnetic sensor, an absolute amount of a variation in geomagnetism, a frequency of a variation in geomagnetism, or information acquired from a pressure sensor signal.

For example, an acceleration sensor can be used as the body motion sensor 300. In this case, the information acquisition unit 110 acquires acceleration information (or position information) as body motion information from an acceleration sensor. In addition, the body motion sensor 300 may be a gyro sensor, an altitude sensor, a geomagnetic sensor, or a pressure sensor. The body motion sensor 300 may be, for example, a global positioning system (GPS) receiver. In this case, the GPS receiver (the body motion sensor 300) acquires positional information indicating a current position of the wearable apparatus 500 (subject) based on radio waves transmitted from GPS satellites and the information acquisition unit 110 acquires positional information regarding the wearable apparatus 500 (subject) as body motion information.

The processing unit 130 generates report information for reporting stamina information indicating stamina of the user based on biometric information. The output unit 170 outputs the report information generated by the processing unit 130 to, for example, a display unit 900. Functions of the information acquisition unit 110, the processing unit 130, and the output unit 170 can be realized by a program or hardware such as various processors (a CPU and the like) and an ASIC (gate array or the like). For example, the processor 400 realizes the functions of the information acquisition unit 110, the processing unit 130, and the output unit 170 in the example of FIG. 1. However, the embodiment is not limited thereto. Various modifications can be made in such a manner that, for example, the information processing system 100 includes a plurality of processors, the function of the processing unit 130 is realized by a first processor, the function of the information acquisition unit 110 is realized by a second processor, and the function of the output unit 170 is realized by a third processor.

The output unit 170 may be, for example, a communication unit. In this case, the output unit 170 is communicatively connected to a report unit such as the display unit 900 in FIG. 1 via a network and communicates the report unit to output report information to the report unit.

Further, the output unit 170 may also be the display unit 900 in FIG. 1 or a report unit such as an audio output unit or a vibration unit (not illustrated) as well as the above-described communication unit. In this case, the output unit 170 outputs report information to report information to the user. For example, in a case in which the output unit 170 is a display unit, the report information is display information. Then, information is reported to the user by outputting the display information to display the display information on a screen of the display unit. For example, in a case in which the output unit 170 is an audio output unit (not illustrated), the report information is audio output information. Then, information is reported to the user when the audio output unit outputs audio based on audio output information. Similarly, for example, in a case in which the output unit 170 is a vibration unit (not illustrated), the report information is vibration information. Then, information is reported to the user when the vibration unit vibrates based on the vibration information.

### 2. Technique According to Embodiment

Next, a technique according to the embodiment will be described. In the embodiment, the processing unit 130 generates report information regarding stamina information (first stamina information) regarding the user as first report information for the user. Further, the processing unit 130 generates at least one piece of report information among second stamina information different from the above-described stamina information in an environment situation, third stamina information for another user, advice information or alert information regarding an activity of the user, and exercise continuable information regarding the user as second report information for the user. Then, the output unit 170 outputs the first report information and the second report information to, for example, the display unit 900 and the display unit 900 displays the acquired first report information and second report information.

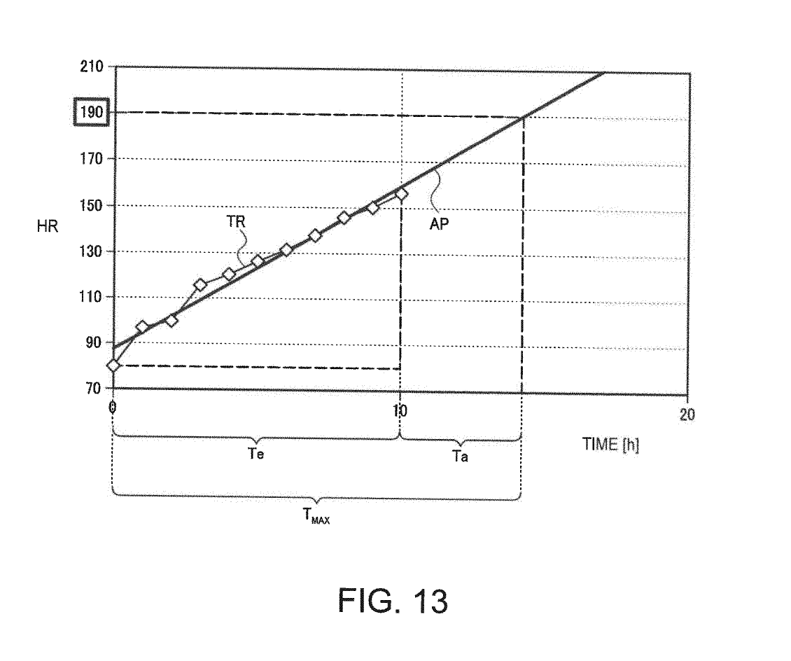

Here, the stamina information is information indicating stamina (endurance) of a user. For example, stamina information may be information indicating a stamina amount consumed in an activity (exercise) of a user (stamina consumption amount) or may be information indicating a stamina amount remaining in a user as a result obtained from an activity (stamina remaining amount). A method of specifying the stamina information will be described below with reference to FIG. 13 and the like.

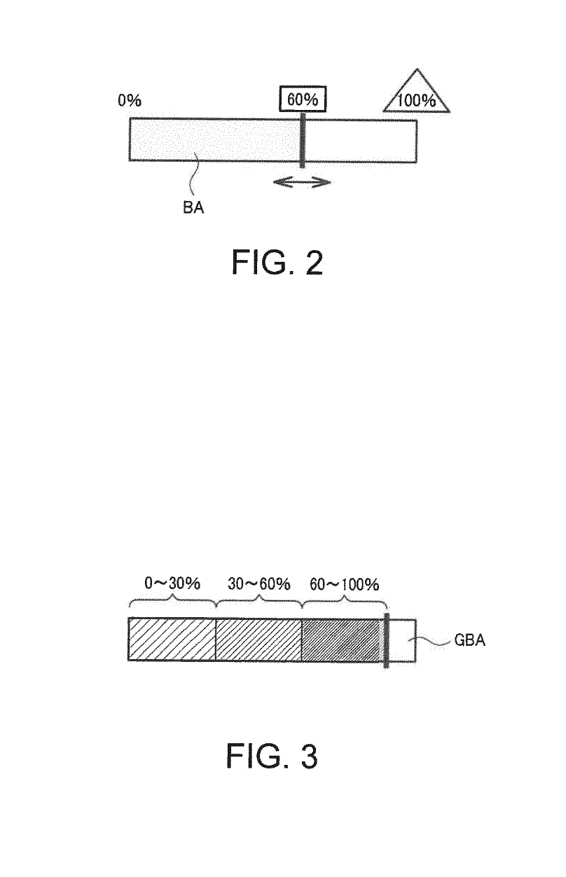

In the embodiment, as in the example of FIG. 2, for example, a stamina consumption amount of the user is expressed with 0% to 100% and an image BA of a horizontal bar graph (bar) indicating which percentage of stamina is consumed in 0% to 100% is displayed on the display unit 900. That is, in the example of FIG. 2, the image BA is report information regarding the stamina information. The image BA indicates that the stamina consumption amount of the user is 60%. The stamina remaining amount can also be displayed in the same way. In the embodiment, the method of displaying the report information regarding the stamina information is not limited to the example of FIG. 2. For example, the report information regarding the stamina information may be displayed in the method illustrated in FIG. 3. In the example of FIG. 3, an image GBA is displayed in which a color of a bar indicating the stamina consumption amount is deeper whenever the stamina consumption amount increases by 30%. That is, a region of 0% to 30% is displayed with a first color, a region of 30% to 60% is displayed with a second color deeper than the first color, and a region of 60% to 100% is displayed with a third color further deeper than the second color. At this time, gradation display in which the color of a bar indicating the stamina consumption amount is gradually deeper from 0% to 100% may be realized.

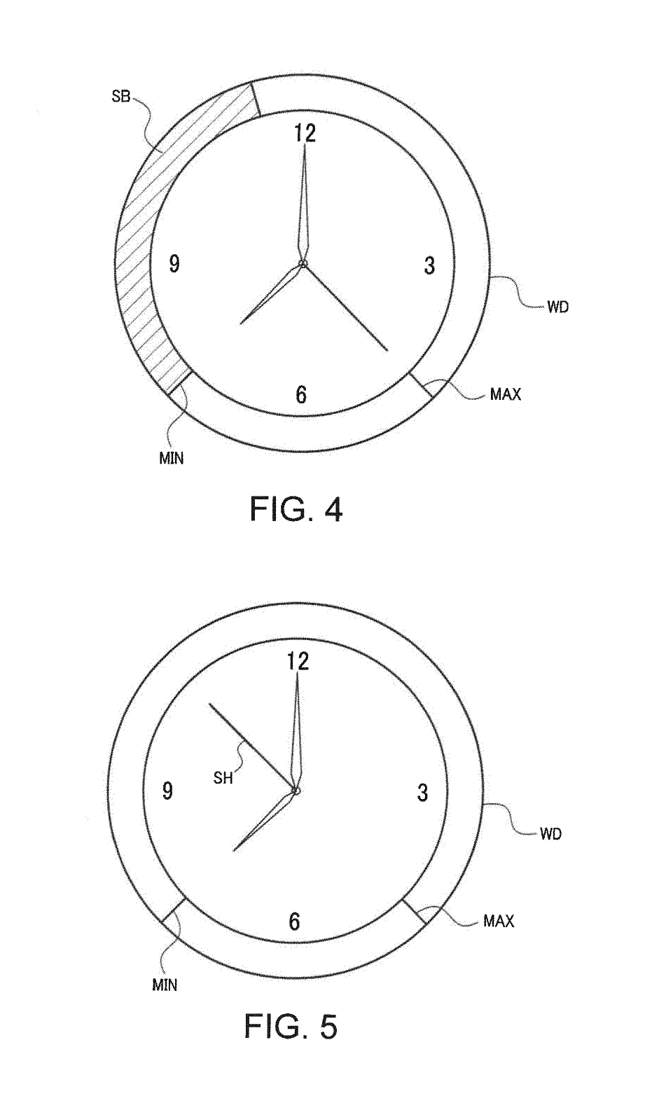

Further, report information of the stamina information may be displayed in a method illustrated in FIG. 4 or 5. In the example of FIG. 4, the display unit 900 is a monitor and displays a dial plate WD and timepiece hands of a timepiece. In the example of FIG. 4, on the display unit 900, an image SB of a bar indicating a stamina consumption amount (or a stamina remaining amount) of a user is displayed in an outer frame of the dial plate WD and the stamina consumption amount is reported to the user by expanding or contracting the image SB of the bar to correspond to the stamina consumption amount of the user. In the example of FIG. 4, the image SB can be expanded or contracted and displayed from the position of MIN indicating that the stamina consumption amount is the minimum to the position of MAX indicating that the stamina consumption amount is the maximum. In the case of FIG. 4, the report information of the stamina information is information of a display image displayed on the display unit 900.

In the example of FIG. 5, the display unit 900 displays a dial plate WD and a second hand SH of a timepiece, and a pattern indicating the stamina consumption amount (or the stamina remaining amount) of a user is printed in an outer frame of the dial plate WD. Specifically, a line MIN indicating that the stamina consumption amount is the minimum and a line MAX indicating that the stamina consumption amount is the maximum are printed. The stamina consumption amount is reported to the user when the second hand SH points a position corresponding to the stamina consumption amount between the line MIN and the line MAX. For example, in the middle of mountain-climbing, a user cares about time up to minutes, but necessity to care about time up to seconds is not so high. Therefore, the second hand SH moves to indicate time at the normal time. The second hand SH moves to indicate stamina information at the time of mountain-climbing. However, the embodiment is not limited thereto and another timepiece hand may be used to indicate stamina information. In the case of FIG. 5, the report information of the stamina information is control information (rotation information) of the second hand SH.

The second report information is at least one piece of report information among second stamina information different from the stamina information indicated by the first report information in an environment situation, third stamina information regarding another user, advice information or alert information regarding an activity of the user, and exercise continuable information regarding the user. The processing unit 130 may generate a plurality of pieces of information among the second stamina information, the third stamina information, the advice information or the alert information, and the exercise continuable information of the second report information, and the output unit 170 may output the plurality of pieces of information. For example, the processing unit 130 may generate the second stamina information and the third stamina information, the output unit 170 may output the second stamina information and the third stamina information to the display unit 900, and the display unit 900 may simultaneously display the second stamina information and the third stamina information.

The second stamina information is information regarding the same user as the first stamina information and refers to stamina information in an environment situation different from that of the first stamina information. The environment situation is an exercise situation of a user or a surrounding environment situation when a user performs an activity. The environment situation is, for example, a place or a time at which a user performs an activity or weather, temperature, atmospheric pressure, or the like when a user performs an activity. The exercise situation indicates, for example, an activity pace of a user such as a walking pace or a running pace, a time (cumulative activity time) in which an activity continues up to the present time, whether a user is moving on a flat or is moving uphill or downhill.

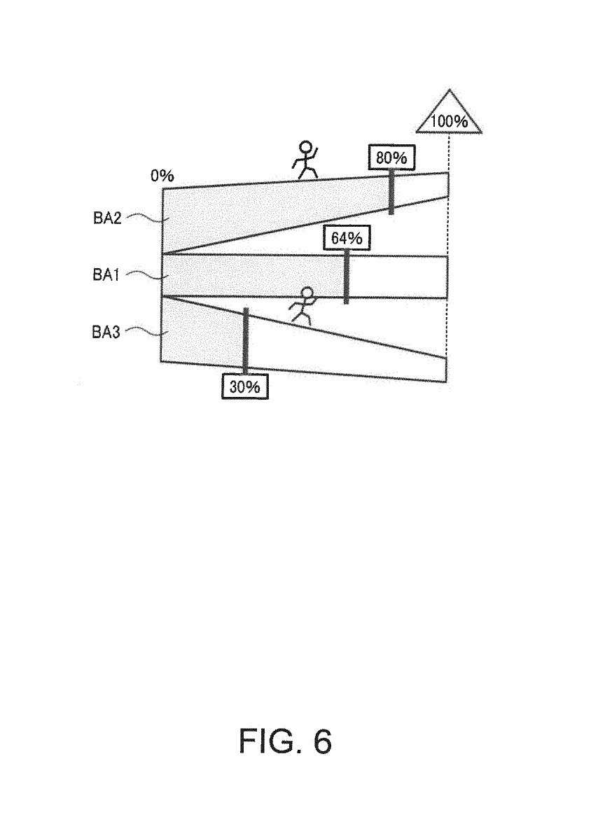

For example, FIG. 6 illustrates an example of a case in which the second report information related to the second stamina information is displayed in addition to the first report information related to the first stamina information. In the example of FIG. 6, an image BA1 indicates the first stamina information and images BA2 and BA3 indicate the second stamina information. In this example, an environment situation is an exercise situation of a user. That is, the exercise situation of the first stamina information is a situation in which a user is moving in a flat. An exercise situation of the second stamina information is a situation in which a user is moving uphill or downhill unlike the exercise situation of the first stamina information. When the environment situation is changed, an estimated value of the stamina consumption amount (or a stamina remaining amount) of the user is also changed before and after the environment situation is changed. For example, stamina is more consumed in a case in which the user is moving uphill than a case in which a user is moving in a flat. Therefore, the stamina remaining amount has to be estimated smaller in a case in which the user is moving uphill than a case in which the user is moving in a flat. In a case in which the user is moving downhill, the stamina consumption amount decreases and the stamina remaining amount increases more than a case in which the user is moving a flat. Therefore, the processing unit 130 generates stamina information in an uphill exercise or stamina information in a downhill exercise as the second stamina information. Further, the output unit 170 outputs the first report information indicating the first stamina information and the second report information indicating the second stamina information. Then, the display unit 900 displays the first report information and the second report information. For example, in the example of FIG. 6, the image BA1 indicates that a stamina consumption amount of the user is 64% in a case in which the user is moving in a flat. The image BA2 indicates that a stamina consumption amount of the user is 80% in a case in which the user is moving uphill. The image BA3 indicates that a stamina consumption amount of the user is 30% in a case in which the user is moving downhill.

Thus, for example, the user can ascertain the stamina information in an uphill exercise or a downhill exercise as well as the stamina information when the user is moving in a flat.

The third stamina information is information indicating stamina of another user different from the user of the first stamina information and the second stamina information.

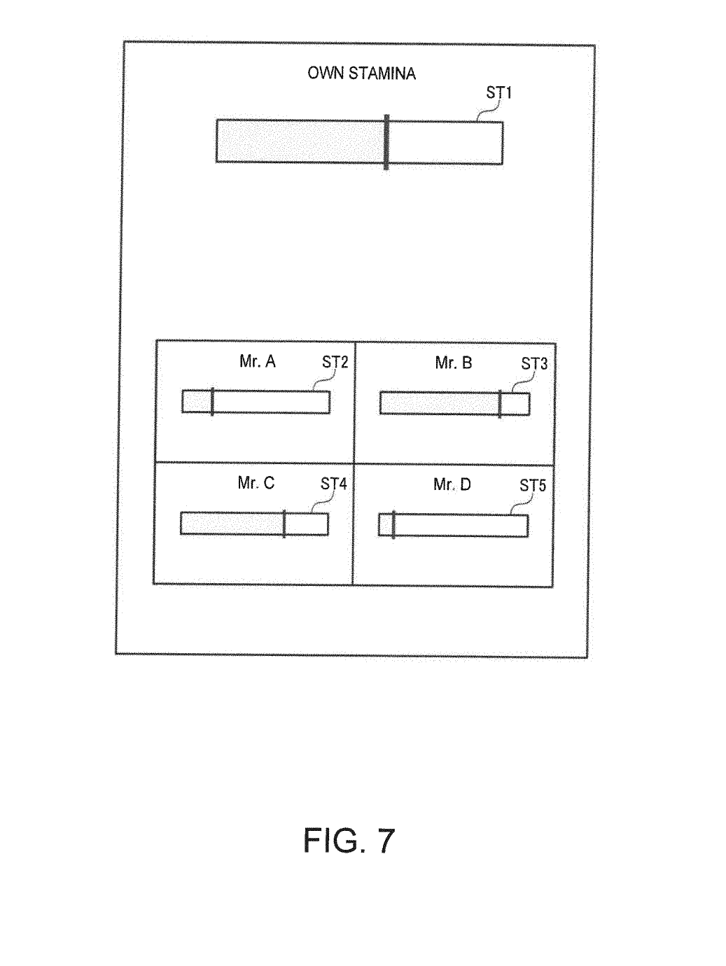

For example, FIG. 7 illustrates an example in which the second report information related to the third stamina information is displayed in addition to the first report information related to the first stamina information. In the example of FIG. 7, the first report information related to the first stamina information is an image ST1 indicating own stamina information, and the second report information related to the third stamina information is images (ST2 to ST5) indicating pieces of stamina information regarding 4 people, Mr. A, Mr. B, Mr. C, and Mr. D. For example, when members of a team climb a mountain and stamina information regarding each member can be confirmed, it is easily to appropriately determine a timing at which the members take a break or confirm whether any member is late among the member.

The advice information is information indicating an advice about an activity of a user and is, for example, recommendation information for recommending an activity to be performed in the future by the user. The recommendation information may include recommendation information regarding not only an activity of a user but also an action of the user such as water supplementation.

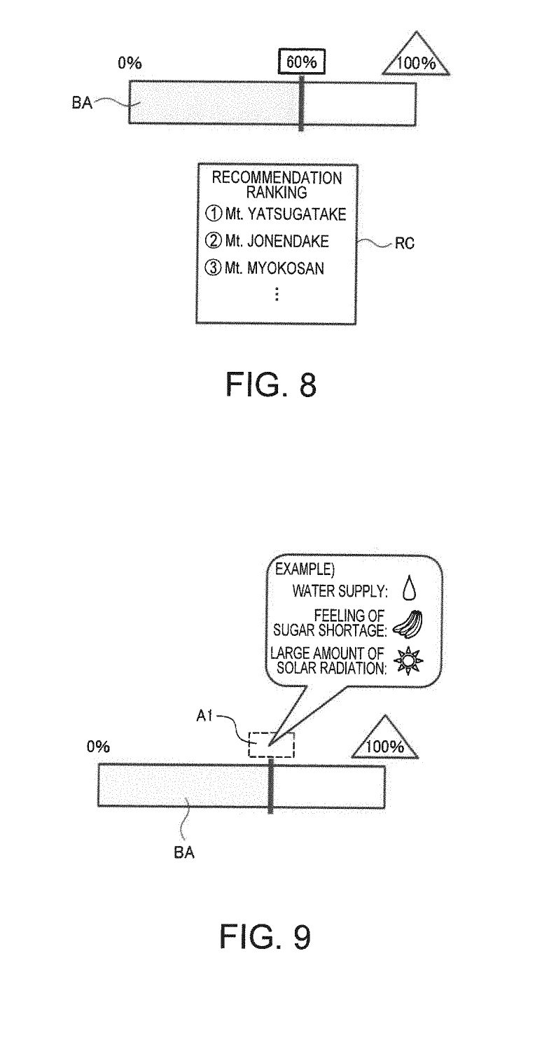

For example, FIG. 8 illustrates an example in which the second report information related to the advice information is displayed in addition to the first report information related to the first stamina information. In the example of FIG. 8, as in the above-described example of FIG. 2, an image BA indicates the first stamina information. For example, an image RC indicates advice information. In the example of FIG. 8, the image RC indicates ranking of mountains recommended to a user. Recommendation ranking shown in the image RC is specified by comparing, for example, pieces of stamina information regarding users, information (altitudes, slopes, or the like) regarding climbed mountains, and information regarding recommendatory mountains to each other.

The alert information is information indicating a warning (alert) of an activity performed by a user. For example, the alert information is information for encouraging the user to stop an activity currently performed by a user or encouraging the user to take a break or water supplement.

For example, FIG. 9 illustrates an example in which the second report information related to the advice information or the alert information is displayed in addition to the first report information related to the first stamina information. In the example of FIG. 9, as in the above-described example of FIG. 2, an image BA indicates the first stamina information. For example, an icon image AI displayed in an upper portion of the image BA indicates advice information or alert information. Specifically, the icon image is, for example, an image for encouraging a user to drink water or an image for warning against sugar shortage, or an image for warning against a large amount of solar radiation.

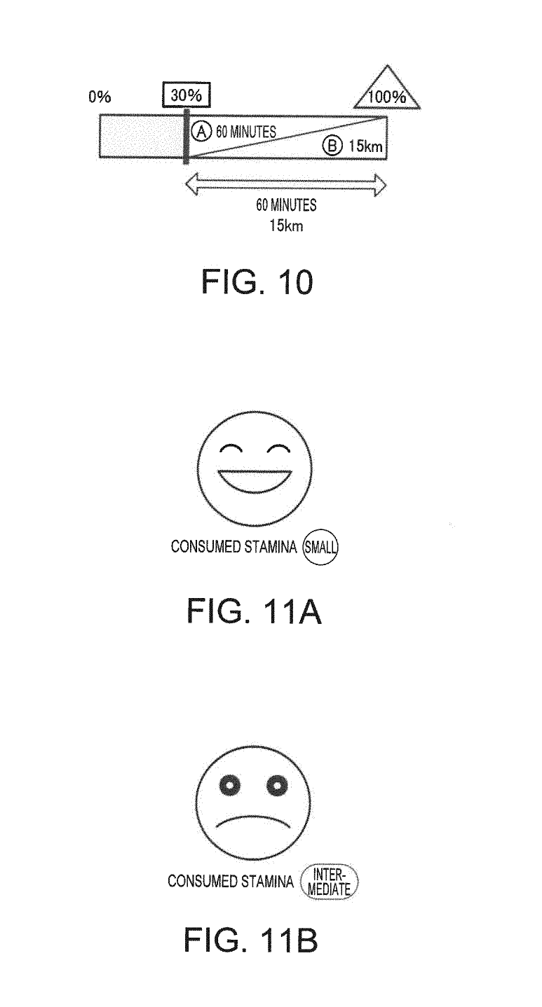

The exercise continuable information is, for example, information indicating a time in which an exercise currently performed by a user can be continued or a movable distance by which an exercise currently performed by a user is continued. For example, as in an example of FIG. 10, the second report information related to an exercise continuable time and a movable distance is displayed on the display unit 900 in addition to the first report information related to the first stamina information. For example, in the example of FIG. 10, a stamina consumption amount of 30% is displayed, an exercise continuable time of 60 minutes and a movable distance 15 km are displayed.

As described above, in the embodiment, the second report information referred to when the user refers to the first report information is displayed on the display unit 900 along with the first report information for reporting the stamina information regarding the user. For example, the user refers to the second report information in conjunction with the first report information. Thus, meaning of the stamina information indicated by the first report information can be delivered to the user more easily. That is, the stamina information regarding the user can be displayed so that the user can easily ascertain the stamina information. Furthermore, assistance can be performed when the user determines an action to be taken subsequently.

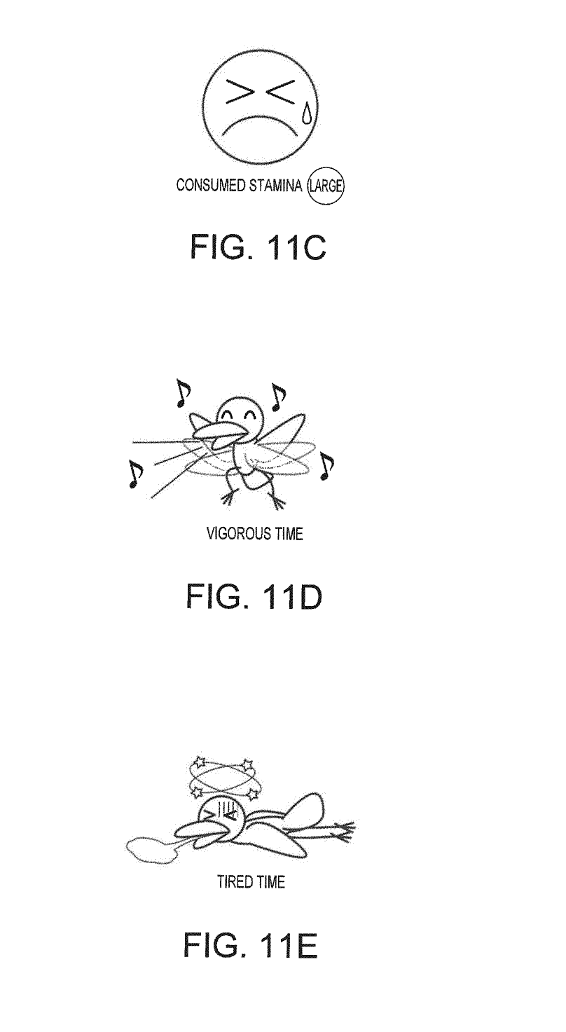

The processing unit 130 may generate information regarding an animation in which a character is used, as the first report information. In this case, the processing unit 130 generates, for example, information regarding animations illustrated in FIGS. 11A to 11E, the output unit 170 outputs the information to the display unit 900, and the display unit 900 displays the animations. For example, the animation in the example of FIG. 11A indicates that a stamina consumption amount is small, the animation in the example of FIG. 11B indicates that a stamina consumption amount is intermediate, and the animation in the example of FIG. 11C indicates that a stamina consumption amount is large. Similarly, the animation in FIG. 11D indicates that the stamina consumption amount is small and a user is vigorous. The animation in FIG. 11E indicates that the stamina consumption amount is large and a user is tired.

Thus, the user can confirm the animation displayed on the display unit 900 and can ascertain an overview of the own stamina information at first glance.

In the embodiment, configurations may be realized as follows. That is, the information processing device 100 includes a memory (the storage unit 150) that stores information (for example, a program or various kinds of data) and the processor 400 (the processing unit 130 or a processor configured with hardware) that operates based on information stored in the memory. The processor 400 acquires biometric information regarding a subject from the biometric sensor 200 (biometric sensor device) installed in the wearable apparatus 500. Then, the processor 400 generates report information regarding stamina information regarding a user as the first report information for the user, generates at least one piece of report information among the second stamina information different from the stamina information in an environment situation, the third stamina information for another user, the advice information or the alert information regarding an activity of the user, and the exercise continuable information regarding the user as the second report information for the user, and outputs the first report information and the second report information.

In the processor (the processing unit 130), the function of each unit may be realized by, for example, individual hardware or the function of each unit may be realized by integrated hardware. The processor may be, for example, a central processing unit (CPU). However, the processor is not limited to a CPU and any of various processors such as a graphics processing unit (GPU) or a digital signal processor (DSP) can be used. The processor may be a hardware circuit realized by an application specific integrated circuit (ASIC). The memory (the storage unit 150) may be a semiconductor memory such as a static random access memory (SRAM) or a dynamic random access memory (DRAM), may be a register, may be a magnetic storage device such as a hard disk device, or may be an optical storage device such as an optical disc device. For example, the memory stores a command which can be read by a computer. Thus, when the command is executed by the processor, the function of each unit of the processing unit 130 is realized. Here, the command may be a command in a command set that forms a program or may be a command by which a hardware circuit of the processor is instructed to perform an operation.

The embodiment can be applied to an information processing method including: acquiring biometric information from a biometric sensor 200; generating report information for reporting stamina information indicating stamina of a user as first report information for the user based on the biometric information; generating at least one piece of report information among the second stamina information different from the stamina information in an environment situation, the third stamina information regarding another user, advice information or alert information regarding an activity of the user, and the exercise continuable information regarding the user, as the second report information for the user; and outputting the first report information and the second report information.

### 3. Process of Specifying Stamina Information

Next, a process of specifying the above-described stamina information will be described. Here, a heart rate will be focused on in the biometric information. For example, a heart rate may considerably increase even when a person with no stamina does an exercise for a short time. In a case in which a person with stamina does the same exercise, an increasing amount of a heart rate is smaller than that of a person with no stamina. Therefore, it is considered that a person whose heart rate is easily changed has no stamina and a person whose heart rate is rarely changed has stamina.

Accordingly, the processing unit 130 estimates stamina information based on a change in the biometric information by an activity continued by a user. Specifically, as a change in a heart rate of a person is larger, a stamina consumption amount is estimated to be larger. As a change in a heart rate of a person is smaller, a stamina consumption amount is estimated to be smaller.

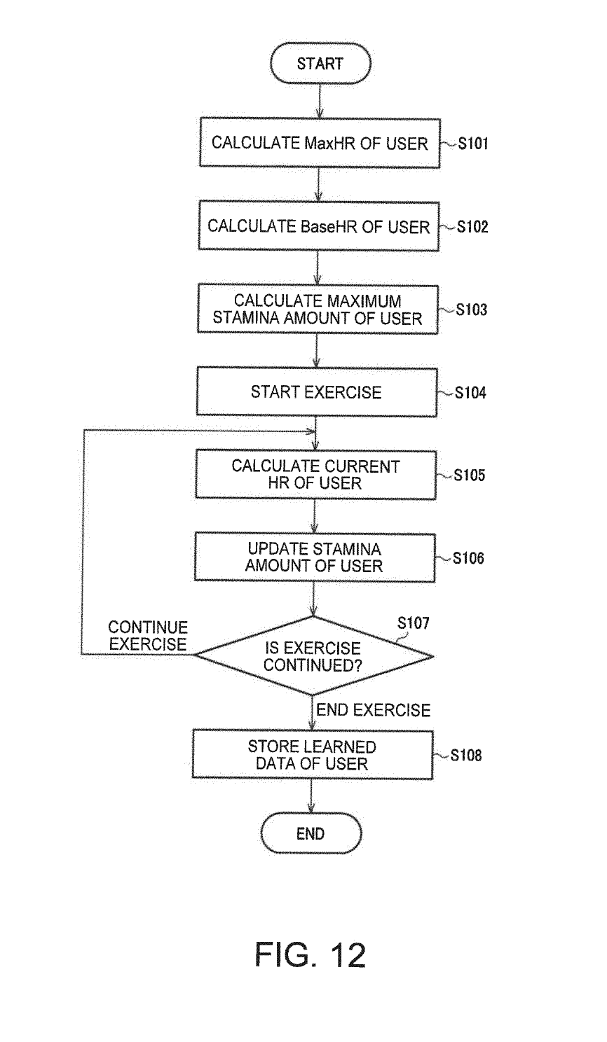

A flow of a specific process is illustrated in the flowchart of FIG. 12. First, the processing unit 130 calculates a maximum heart rate MaxHR of a user (exerciser) by Equation (1) below (S101). In Equation (1) below, age is an age of the user.

MaxHR=(220−age) (1)

Subsequently, the processing unit 130 calculates a heart rate BaseHR at a resting time of (normal time) the user based on a past exercise history of the user stored in the storage unit 150 or body information such as age and weight (S102).

Further, the processing unit 130 calculates a maximum stamina amount of the user (S103). Here, the maximum stamina amount is, for example, at least one piece of information between information indicating a maximum time (maximum activable time T_{MAX}) which can be utilized by the user using the resting time (normal time) as a starting point and a maximum distance (maximum movable distance) in which the user can move as much as possible. The maximum stamina amount may be obtained in each of the above-described different environment situations. That is, a maximum stamina amount in a case in which it is assumed that a user is moving a flat, a maximum stamina amount in a case in which it is assumed that a user is moving uphill, and a maximum stamina amount in a case in which it is assumed that a user is moving downhill may be obtained.

Here, an example in which the maximum stamina amount is calculated will be described with reference to FIG. 13. In the example of FIG. 13, it is assumed that a user is 30 years old and a heart rate BaseHR at the resting time is 80. A maximum heart rate MaxHR is calculated to 190 using Equation (1) described above. In this case, in the graph of FIG. 13, a transition TR of the heart rate HR when the user continues an exercise for 10 hours is shown. In the graph of FIG. 13, the horizontal axis (X axis) represents a time (h) and the vertical axis (Y axis) represents a heart rate HR.

The transition TR of the heart rate HR at this time is located as an approximate curve AP. An X axis coordinate value when a Y axis coordinate value of the approximate curve AP is 190 (=MaxHR) is assumed to be a maximum activable time T_{MAX}. The maximum movable distance can be obtained by calculating a product of the maximum activable time T_{MAX} and an average movement distance of the user. The average movement distance of the user may be stored in advance in, for example, the storage unit 150 or may be calculated based on body motion information obtained from the body motion sensor 300.

Then, when the user starts an exercise (9104), the processing unit 130 calculates a current heart rate HR of the user (S105). In step 1S05, in a case in which a heart rate can be measured by the biometric sensor 200, the processing unit 130 uses the measured heart rate. Conversely, in a case in which the heart rate may not be measured by the biometric sensor 200, the processing unit 130 estimates a heart rate based on the body motion information acquired from the body motion sensor 300, as will be described below. A heart rate estimation process will be described in detail below.

Then, the processing unit 130 updates the stamina amount (stamina information) of the user based on the maximum stamina amount and the current heart rate or the estimated heart rate (S106). Specifically, a cumulative exercise time Te can be obtained based on the current heart rate or the estimated heart rate, as in FIG. 13. Further, by subtracting the cumulative exercise time Te from the maximum activable time T_{MAX}, it is possible to obtain an exercise continuable time Ta as a stamina remaining amount. The stamina amount may be obtained in each of the above-described different environment situations, as in the maximum stamina amount.

Next, the processing unit 130 determines whether the user continues the exercise (S107). In a case in which it is determined that the user continues the exercise (the exercise is continued in S107), the process returns to step S105. In a case in which it is determined that the user does not continue the exercise (the exercise ends in S107), learned data of the user is stored in the storage unit 150 (S108) and the process ends. The learned data is biometric information such as a heart rate of the user, body motion information, coefficient information of Equation (5) to be described below, or the like.

Thus, stamina information can be estimated by adding a change in the biometric information in a case in which the user continues an activity. As a result, it is possible to estimate the stamina information in which an individual difference between users is reflected.

Next, a modification example of the process of specifying stamina information will be described. In the modification example, the processing unit 130 performs an estimation process of estimating biometric information in a case in which the information acquisition unit 110 may not acquire biometric information from the biometric sensor 200. Then, the processing unit 130 estimates stamina information based on coefficient information used in a biometric information estimation process.

Thus, even when the biometric information may not be acquired from the biometric sensor 200, the stamina information regarding the user (subject) can be reported (suggested) to the user.

Specifically, for example, in a case in which the information acquisition unit 110 may not acquire the biometric information regarding the subject from the biometric sensor 200, the processing unit 130 estimates the biometric information regarding the subject based on the body motion information acquired from the body motion sensor 300. Then, the processing unit 130 obtains an activity strength of the subject and an activity time corresponding to the activity strength based on the body motion information and estimates the biometric information based on the obtained activity time. In this way, by using the activity strength and the activity time corresponding to the activity strength in the biometric information estimation process, it is possible to reflect active content of the subject in a result of the biometric information estimation process more accurately.

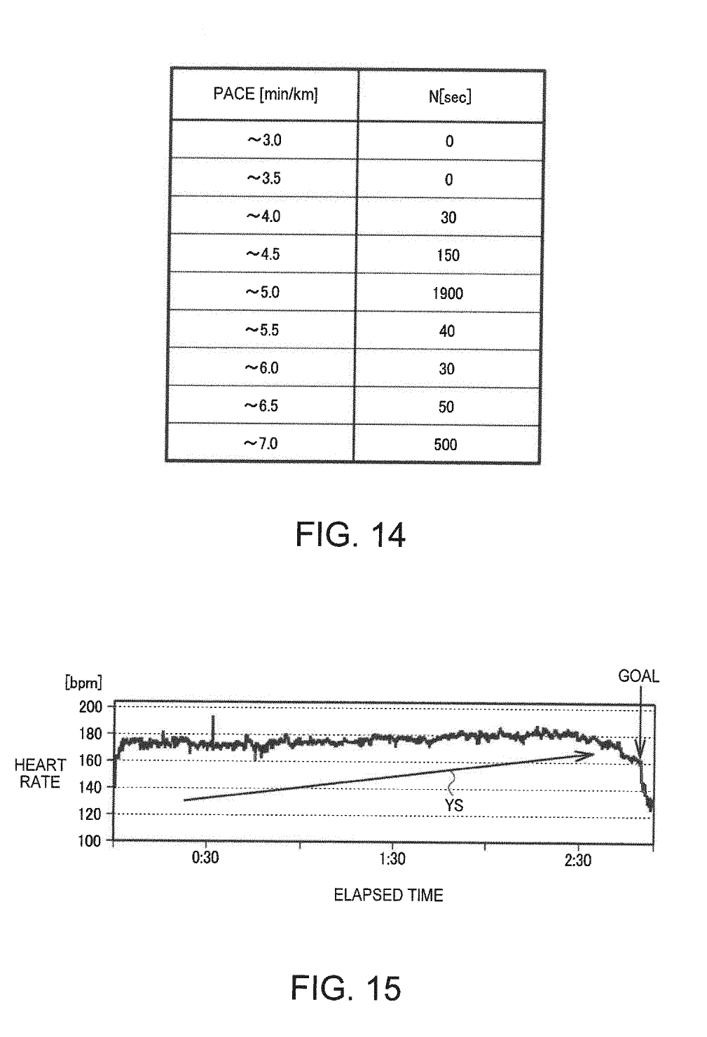

Here, the activity strength is, for example, a numerical value or the like indicating the strength of an activity performed by the subject and is specifically a pace (min/km) at a running time (moving time), as illustrated in FIG. 14. The activity time corresponding to the activity strength is a time in which an activity of the activity strength is performed. For example, in the example of FIG. 14, a pace (min/km) at the running time is partitioned into a predetermined ranges and each range of the running pace is set as a different activity strength. For example, a running pace up to 3.0 (min/km) is set as a first activity strength and a running pace of 3.0 (min/km) to 3.5 (min/km) is set as a second activity strength. Next, in a running pace partitioned at intervals of 0.5 (min/km) in this way, a first activity strength to a ninth activity strength are set as activity strengths with different levels. In the example of FIG. 14, each cumulative activity time N (sec) at the time of performing an activity of each activity strength in the nine activity strengths is measured. For example, an activity time corresponding to the fourth activity strength which is a running pace of 4.0 (min/km) to 4.5 (min/km) is 150 (sec). An activity time corresponding to the fifth activity strength which is a running pace of 4.5 (min/km) to 5.0 (min/km) is 1900 (sec).

Then, in a case in which the information acquisition unit 110 can acquire the biometric information from the biometric sensor 200, the processing unit 130 estimates the stamina information using the biometric information measured by the biometric sensor 200 and generates the report information of the stamina information. The output unit 170 outputs the generated report information to the display unit 900 and the display unit 900 displays the report information related to the stamina information. At this time, the biometric information measured by the biometric sensor 200 or the estimated biometric information may be displayed additionally on the display unit 900. Thus, while the user wearing the electronic apparatus 500 (FIG. 23) according to the embodiment is performing an activity, it is possible to confirm the stamina information.

Meanwhile, when a heart rate is estimated based on the body motion information, as described above, for example, in a case in which an exercise is continued for a long time, it is necessary to notice that a heart rate is not constant even when the exercise is performed under the same conditions. For example, an example is illustrated in FIG. 15. The graph illustrated in FIG. 15 is a graph indicating a heart rate measured at the time of running a marathon at a substantially constant pace. The horizontal axis represents an elapsed time and the vertical axis represents a heart rate (bpm: beats per minute). In the example of FIG. 15, even when a user is continuously running at a substantially constant pace, the heart rate gradually increases from start to goal, as indicated by an arrow YS. For example, the heart rate is around 170 bpm when 30 minutes are elapsed from the start. The heart rate is 180 bpm or greater after 2 hours from the start. This is because a heartbeat drift phenomenon occurs in a human body. The heartbeat drift phenomenon is a phenomenon in which a heart rate gradually increases when the same exercise load is continuously applied in a human body. A mechanism in which this phenomenon occurs is not completely solved, but it is pointed out that occurrence of a dehydration symptom is a main cause. When the exercise is continued for a long time and the heart rate may not be measured, an estimated heart rate may be diverged from an actual heart rate in a case in which the heart rate is estimated. Accordingly, in the embodiment, estimation precision of a heart rate is improved by simultaneously analyzing how long the exercise load is continued in addition to the exercise load itself. Thus, it is possible to improve the estimation precision when the stamina information is obtained based on the heart rate. The exercise load is the above-described activity strength and the time in which the exercise load is continued is an activity time corresponding to the above-described activity strength.

Specifically, the processing unit 130 according to the embodiment estimates the biometric information based on the activity time corresponding to the activity strength and coefficient information corresponding to the activity time to be described below. In other words, the processing unit 130 learns correlation between the activity time corresponding to the activity strength and the heart rate which is biometric information and performs a heart rate estimation process based on a learning result. At this time, the information obtained as the learning result is equivalent to the coefficient information corresponding to the activity time. Then, the processing unit 130 estimates the stamina information based on the coefficient information used for the estimation process. A process of estimating a heart rate as biometric information will be described below, but the embodiment is not limited thereto. Biometric information other than the heart rate may be estimated.

For example, in a specific example to be described below, a heart rate is estimated using each activity strength illustrated in FIG. 14 described above and each activity time corresponding to each activity strength. The heart rate estimation process is performed broadly in the following three steps. First, a standard heart rate serving as a standard is calculated from the activity strength. Next, a correction amount of the standard heart rate is calculated from the activity time corresponding to the activity strength. Then, an estimated heart rate is obtained by adding the standard heart rate and the correction amount as in Equation (2) below.

pulse=pulseᵢₙᵢ+f(M)+p(S) (2)

Here, the standard heart rate is a heart rate in a state in which the heartbeat drift phenomenon does not occur at all. In Equation (2) above, a value expressed with pulseᵢₙᵢ+f(M) corresponds to the standard heart rate. Then, an estimated heart rate pulse is obtained by adding an influence of the heartbeat drift phenomenon as the form of a correction amount p(S) to the standard heart rate. In Equation (2), pulseᵢₙᵢ is, for example, a heart rate at a resting time (normal time), M=(M₁, M₂, . . . )εRm is information indicating an activity strength, and S=(S₁, S₂, . . . )εRm is information indicating an activity time. Specifically, in the example of FIG. 14, M₁ is information indicating a pace of 3.0 (min/km) and S₁ is information indicating a cumulative activity time in which an activity is performed at a pace of 3.0 (min/km). The same also applies to the other M₂ to MS and S₂ to S₉. When it is assumed that each piece of information is obtained through linear Bayesian estimation, the estimated heart rate pulse can be expressed in the form of linear combination expressed in Equation (5) below by setting f(M) to Equation (3) below and setting p(S) to Equation (4). In Equations (3) to (5), wₖ is coefficient information for an activity strength and wₖ₊ₘ is coefficient information for an activity time. Therefore, by learning and updating the coefficients wₖ and wₖ₊ₘ, it is possible to express correlation among the estimated heart rate, and the activity strength and the activity time. Further, Φ is a predetermined conversion function.

$$\begin{matrix}f ( M ) = {\sum }_{k = 1}^{m} {w}_{k} {ϕ}_{k} ( {M}_{k} ) & ( 3 ) \\ p ( S ) = {\sum }_{k = 1}^{s} {w}_{k + m} {ϕ}_{k} ( {S}_{k} ) & ( 4 ) \\ pulse = {pulse}_{im} + {\sum }_{k = 1}^{m} {w}_{k} {ϕ}_{k} ( {M}_{k} ) + {\sum }_{k = 1}^{s} {w}_{k + m} {ϕ}_{k} ( {S}_{k} ) & ( 5 )\end{matrix}$$

Thus, it is possible to reflect the activity time and the activity strength of the user in the stamina information. Further, for example, it is possible to add an increase in the heart rate by the heartbeat drift phenomenon and estimate the heart rate. In the estimation of the coefficient information and the biometric information, another technique such as a Kalman filter can also be used as well as the above-described linear Bayesian estimation.

The estimation of the biometric information can be summarized as follows using Equations (3) to (5). That is, as illustrated in FIG. 14, the processing unit 130 specifies each of first to N-th activity times corresponding to each of first to N-th activity strengths of the subject (where N is an integer equal to or greater than 2). Then, the processing unit 130 estimates each of first to N-th pieces of coefficient information corresponding to each of the first to N-th activity times and estimates the biometric information based on each activity time and each piece of coefficient information corresponding to each activity time. In the example of FIG. 14, w₁ to w₉ which are pieces of coefficient information of the activity strengths and w₁₀ to w₁₈ which are pieces of coefficient information of the activity times are estimated and the biometric information is estimated.

Thus, for example, in a case in which the biometric information regarding the subject may not be acquired, an increase in the heart rate by the heartbeat drift phenomenon can be added and the heart rate can be estimated even when an activity of a different activity strength is performed.

The coefficient information wₖ+m of the activity time can be said to be information corresponding to the stamina consumption amount of the subject. Therefore, based on the coefficient information wₖ₊ₘ of the activity time in Equation (5) described above, the processing unit 130 obtains the stamina information indicating stamina consumed by the subject. For example, in this example, the processing unit 130 may obtain a percentage value converted from the coefficient information wₖ₊ₘ as, for example, a stamina consumption amount. A stamina remaining amount of the subject can also be estimated based on a stamina amount estimated to be originally owned by the subject and the estimated stamina consumption amount.

Thus, for example, it is possible to present the stamina information to which the influence of the heartbeat drift phenomenon is added, to the user.

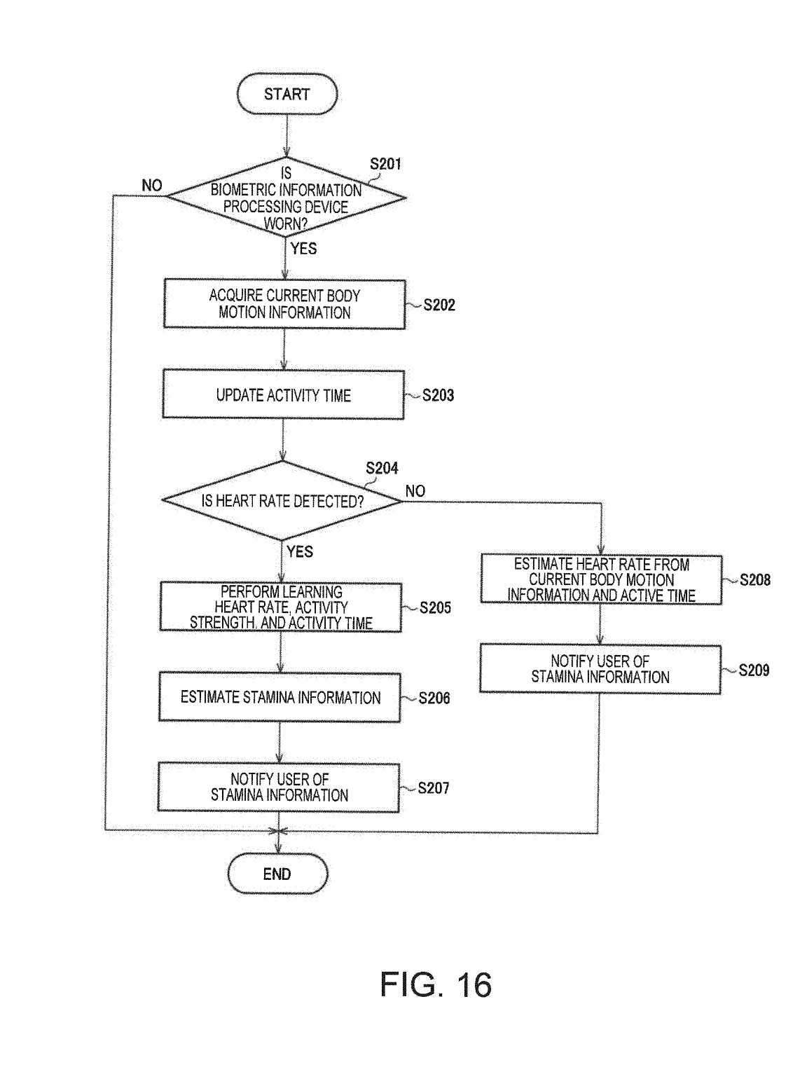

Next, the flow of a process according to the embodiment will be described with reference to the flowchart of FIG. 16. First, the processing unit 130 determines whether the information processing device 100 (FIG. 1) or the electronic apparatus 500 (FIG. 23) including at least the body motion sensor 300 is worn by the subject (S201). When the processing unit 130 determines that the information processing device 100 or the electronic apparatus 500 is not worn by the subject (NO in S201), the process ends.

Conversely, when the processing unit 130 determines that the information processing device 100 or the electronic apparatus 500 is worn by the subject (YES in S201), the information acquisition unit 110 acquires the body motion information from the body motion sensor 300 (S202). Then, based on the acquired body motion information, as illustrated in FIG. 14, the processing unit 130 updates each activity time at each activity strength of the subject (S203).

Next, the processing unit 130 determines whether the heart rate is normally measured by the biometric sensor 200 (S204). For example, in a case in which the information processing device 100 or the electronic apparatus 500 includes a temperature sensor (not illustrated) along with the biometric sensor 200 and the body motion sensor 300, the heart rate is determined to be normally measured by the biometric sensor 200 only in a case in which a temperature output from the temperature sensor is equal to or greater than a given temperature.

Then, in a case in which the processing unit 130 determines that the heart rate is normally measured (YES in S204), the processing unit 130 performs learning using the detected heart rate, the activity strength, and the activity time corresponding to the activity strength (S205). That is, here, the pieces of coefficient information wₖ and wₖ₊ₘ in Equation (5) described above are updated. Next, the processing unit 130 obtains the stamina information based on the updated coefficient information wₖ₊ₘ (S206). For example, the processing unit 130 performs a process of converting the coefficient information wₖ₊ₘ into a stamina consumption amount or the stamina remaining amount. Further, the processing unit 130 notifies the user of the report information of the estimated stamina information (the stamina consumption amount, the stamina remaining amount, or the like) and the heart rate measured by the biometric sensor 200 (S207), and then the process ends.

Conversely, in a case in which the processing unit 130 determines that the heart rate is not normally measured (NO in S204), the processing unit 130 estimates the heart rate according to Equation (5) described above using the activity strength and the activity time corresponding to the activity strength (S208). Then, the processing unit 130 displays the report information of the stamina information estimated in step S206 and the estimated heart rate on the display unit 900 and notifies the user of the report information and the estimated heart rate (S209). Then, the process ends. In this way, even in a case in which the heart rate may not be measured by the biometric sensor 200 by notifying the user of the estimated stamina information and the heart rate, it is possible to prevent the user from not confirming the stamina information and the heart rate at all.

In step S209 of FIG. 16, the stamina information estimated in step S206 is displayed without change. However, in a case in which the information acquisition unit 110 may not acquire the biometric information from the biometric sensor 200, the processing unit 130 may estimate the stamina information using the estimated biometric information and may display the report information of the re-estimated stamina information on the display unit 900. While the biometric information may not be acquired, the biometric information is changed in some cases. In this case, this is because it is considered that the stamina information is also changed. For example, while the heart rate may not be measured, the stamina remaining amount decreases as long as the heart rate increases. Accordingly, by re-estimating the stamina information using the estimated biometric information, for example, it is possible to improve estimation precision of the stamina information.

Additionally, the stamina information can be estimated using the following method. For example, even in a case in which a subject doing an exercise stops the exercise, a heart rate does not return to a normal value instantly. In many cases, it is determined that there is stamina as a time taken to return the heart rate to the normal value is longer. Depending on an exercise load, the heart rate does not return to a normal value and is maintained as a certain high numerical value in some cases even when an exercise is stopped and a certain time is elapsed.

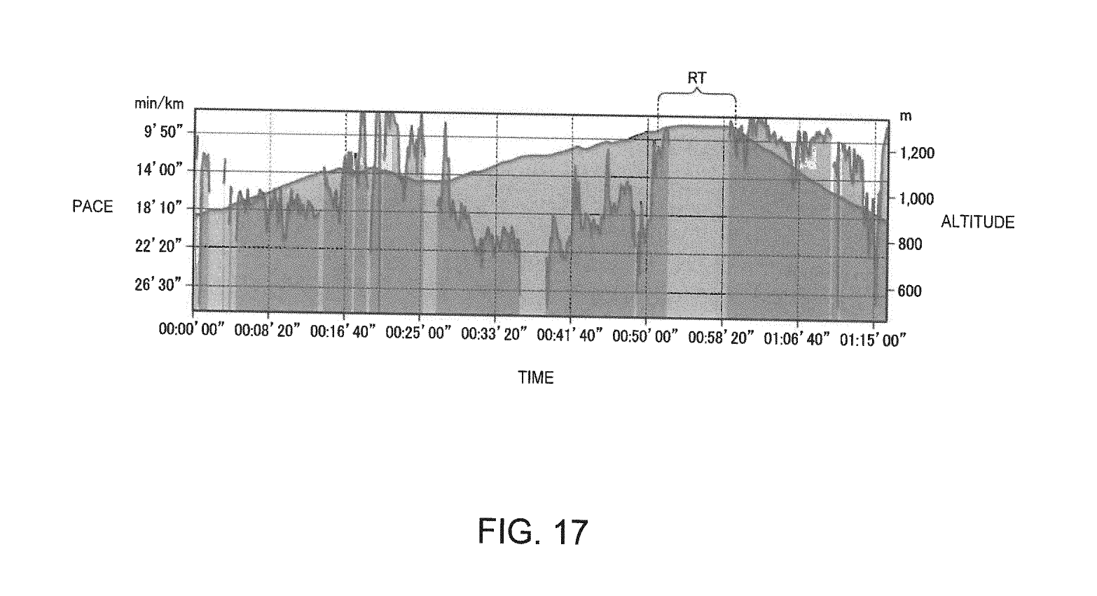

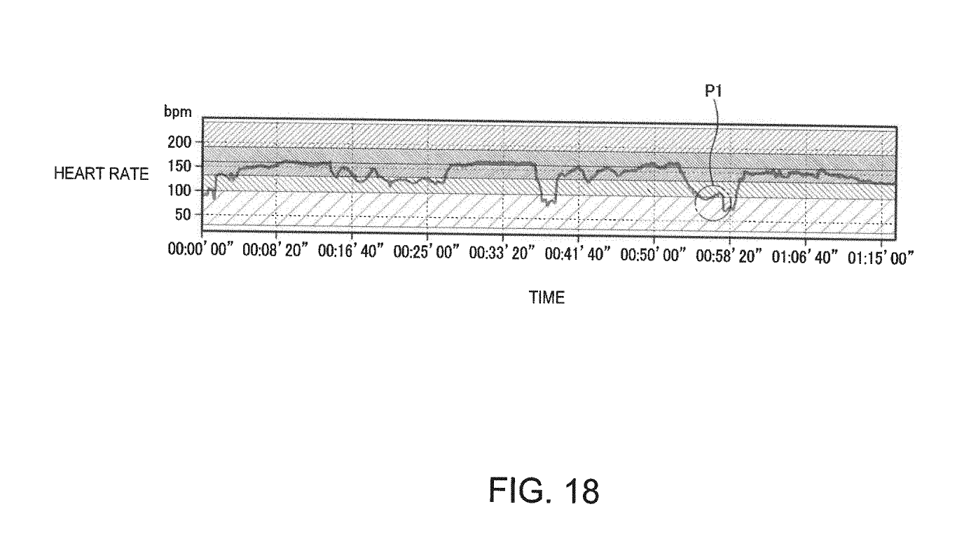

For example, the graph of FIG. 17 shows an example of a relation between a movement pace, an altitude, and an elapsed time when a subject is climbing a mountain. In the graph of FIG. 17, the horizontal axis represents an elapsed time from start of an exercise, the vertical axis on the left side represents a movement pace (min/km), and the vertical axis on the right side represents an altitude (m) of a site at which a subject is located. The graph of FIG. 18 shows a relation between a heart rate and an elapsed time when the subject is performing an activity shown in the graph of FIG. 17. In the graph of FIG. 18, the horizontal axis represents an elapsed time from start of an exercise and the vertical axis represents a heart rate (bpm). The elapsed time of the graph of FIG. 17 is the same as the elapsed time of the graph of FIG. 18.

In the graph of FIG. 17, the subject takes a break for a period indicated by RT. However, referring to FIG. 18, a heart rate does not return to a heart rate at the normal time (here, 55 to 60 is assumed) for a period indicated by P1 regardless of 52′ 32″ at which the subject stops the exercise.

Accordingly, in a modification example, when a heart rate is determined to be stabilized after an exercise is to be determined to be stopped, the degree of consumption of stamina of a subject is obtained using a heart rate at that time (for example, 98 is assumed in the example of FIG. 18).

That is, in the modification example, the information acquisition unit 110 acquires post-activity biometric information from the biometric sensor 200 after the activity of a subject is stopped and a predetermined period is elapsed. Then, the processing unit 130 specifies stamina information indicating stamina consumed by the subject when the subject performs the activity based on the post-activity biometric information.

Specifically, in the modification example, the coefficient information wₖ₊ₘ of the activity time in Equation (5) described above is used as information corresponding to stamina information. In the modification example, stamina information in which the heartbeat drift phenomenon is considered is obtained based on a heart rate when pulses are determined to be stabilized at the time of stopping the activity. Therefore, the coefficient information wₖ₊ₘ is updated to w_{k+m, new} by Equation (6) below. In Equation (6), pulse_{break} is a heart rate when pulses are determined to be stabilized at the time of stopping an activity. For example, pulse_{break} is 98 in the example of FIG. 18. In Equation (6), Time indicates a time from start of an exercise to taking of a break. For example, Time is 52′ 32″ in the example of FIG. 18.

$${w}_{k + m , new} = \frac{{pulse}_{break} - {pulse}_{ini}}{Time} \qquad ( 6 )$$

Then, the obtained w_{k+m, new} is converted into a stamina remaining amount or a stamina consumption amount. Thus, after the activity of the user is stopped, the post-activity biometric information can be added, for example, to estimate stamina information. Further, even in a case in which there is a period in which the biometric information may not be acquired from the biometric sensor 200, for example, estimation precision of the stamina information can be further improved.

In a case in which the estimated stamina information is considerably diverged from an actual stamina of the user, the user may feel a sense of discomfort in the stamina information displayed on the display unit 900. In this case, as stamina is closer to stamina assumed subjectively by the user, it is desirable to correct the stamina information.

Accordingly, the processing unit 130 may receive stamina correction information for correcting stamina information from the user and may correct the stamina information based on the stamina correction information.

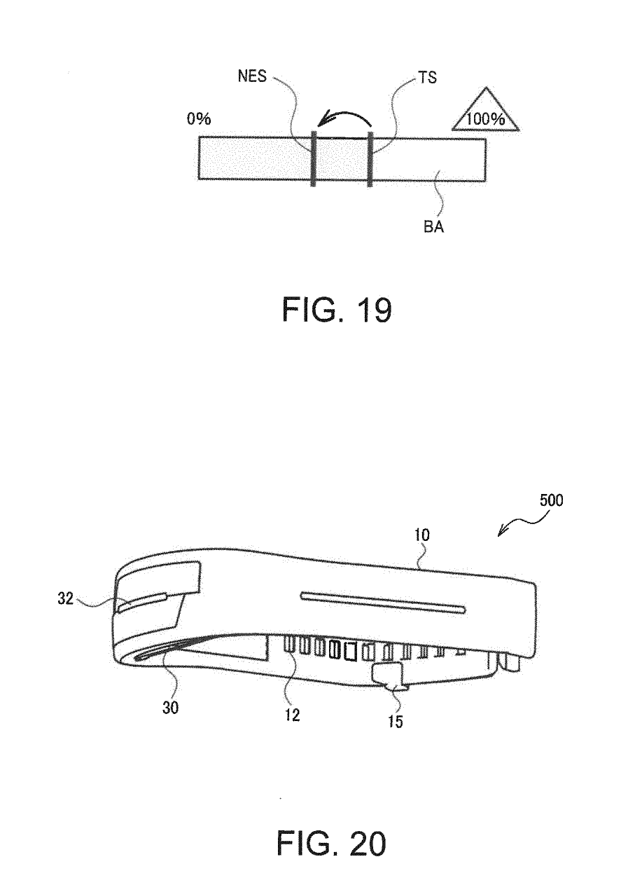

For example, a specific example is illustrated in FIG. 19. In the example of FIG. 19, stamina information estimated by the information processing device 100 is displayed with an image BA. Specifically, stamina of a user is consumed up to a ratio at which a bar image TS is located between 0% and 100% of the image BA. Then, when the user manipulates a manipulation unit (not illustrated), the user can slide the bar image TS in a range of 0% to 100% of the image BA. The manipulation unit may be, for example, a touch panel integrated with the display unit 900 or may be a button or the like. Then, the user slides the bar image TS in the range of 0% to 100% so that a stamina consumption amount is a stamina consumption amount assumed by the user. For example, the bar image TS is slid to a location indicated by NES. Thereafter, the processing unit 130 reads the stamina consumption amount corrected by the user based on the location NES of the slid bar image TS and updates the stamina consumption amount to a read value.

Here, the stamina correction information is stamina information input by the user. More specifically, the stamina correction information is a slide amount of the bar image TS, a display location information of the bar image TS, or manipulation information of the manipulation unit in the example of FIG. 19. The stamina correction information is not limited thereto. For example, a numerical value indicating a specific stamina remaining amount or a stamina consumption amount may be used.

Thus, it is possible to display the report information of the stamina information closer to the stamina assumed subjectively by the user.

### 4. Specific Examples of Wearable Apparatuses

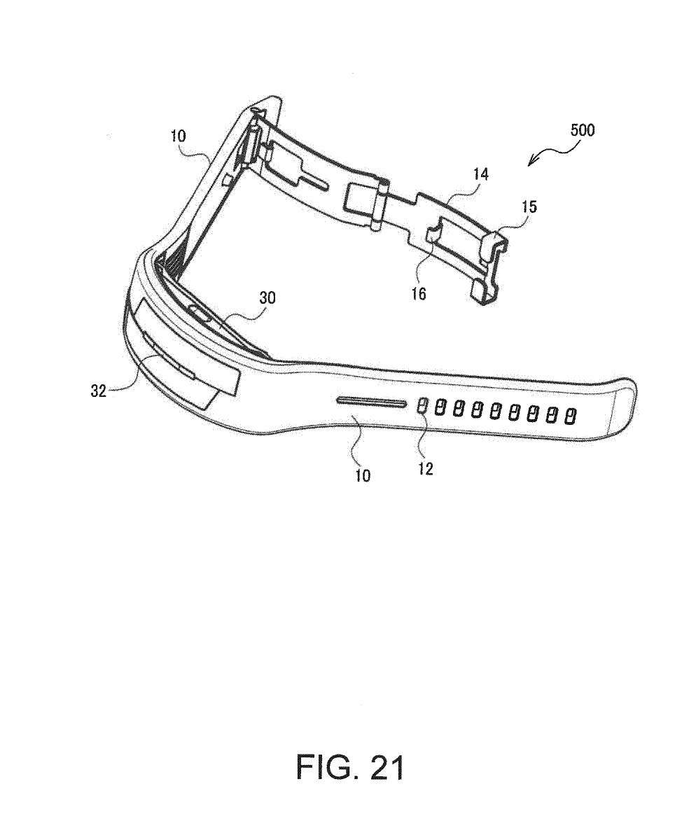

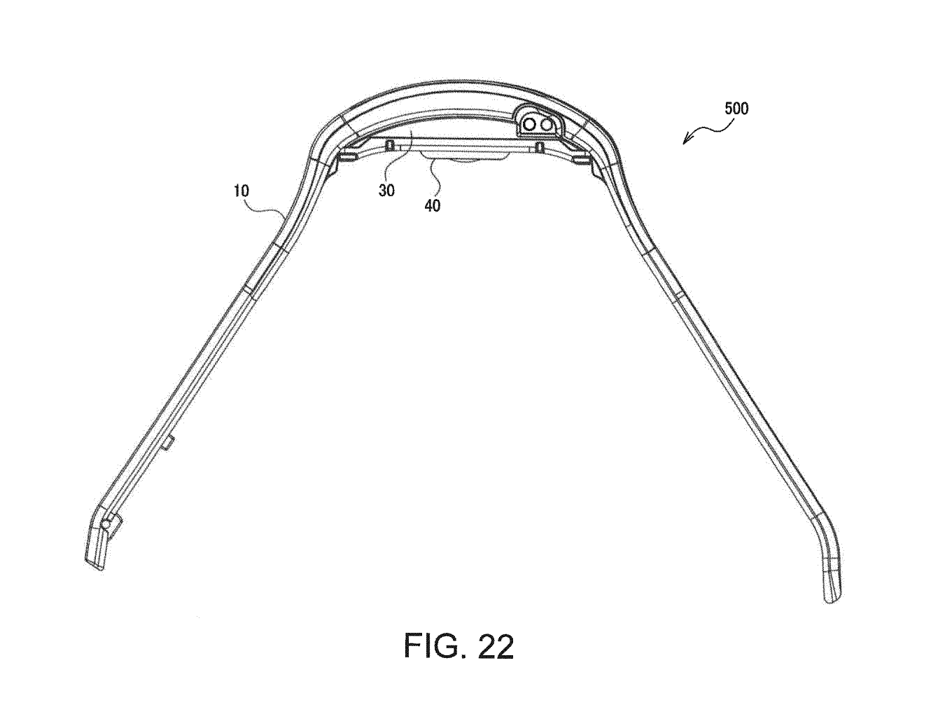

FIGS. 20 to 22 illustrate an example of the outer appearance of the wearable apparatus 500 (wearable apparatus) that acquires biometric information and body motion information. The wearable apparatus 500 according to the embodiment includes a band unit 10, a case unit 30, and a sensor unit 40. As illustrated in FIGS. 20 and 21, the case unit 30 is mounted on the band unit 10. As illustrated in FIG. 22, the sensor unit 40 is installed in the case unit 30 and includes the biometric sensor 200 and the body motion sensor 300 described above. The sensor unit 40 includes the biometric sensor 200 and the body motion sensor 300 in FIG. 1.

The band unit 10 is used to mount the wearable apparatus 500 around a wrist of a user. The band unit 10 includes band holes 12 and a buckle unit 14. The buckle unit 14 has a band insertion portion 15 and a protrusion portion 16. The user wears the wearable apparatus 500 around his or her wrist by inserting one end side of the band unit 10 into the band insertion portion 15 of the buckle unit 14 and inserting the protrusion portion 16 of the buckle unit 14 into the band hole 12 of the band unit 10. The band unit 10 may be configured to include a clasp unit instead of the buckle unit 14.

The case unit 30 is equivalent to a body unit of the wearable apparatus 500. Various constituent elements of the wearable apparatus 500 such as the sensor unit 40 or a circuit substrate (not illustrated) are installed inside the case unit 30. That is, the case unit 30 is a casing that accommodates these constituent elements.

A light-emitting window unit 32 is installed in the case unit 30. The light-emitting window unit 32 is formed of a light-transmission member. A light-emitting unit serving as an interface mounted on a flexible substrate is installed in the case unit 30. Thus, light from the light-emitting unit is exited to the outside of the case unit 30 via the light-emitting window unit 32. Instead of the light-emitting unit, a display unit such as a liquid crystal display (LCD) may be installed or a display unit and a light-emitting unit may be installed together in the case unit 30.

As illustrated in FIG. 23 and the like, the wearable apparatus 500 is worn on a wrist of the user and measure biometric information or body motion information in the worn state.

### 5. Specific Realization Example of Information Processing System

Next, an example of a specific apparatus realizing the information processing system 100 according to the embodiment will be described. A function of the information processing system 100 according to the embodiment may be realized by, for example, the server system 600. An example of this case is illustrated in FIG. 23. For example, the information processing system 100 which is the server system 600 is connected to the wearable apparatus 500 (electronic apparatus) via a network NE and acquires biometric information and body motion information regarding subject from the wearable apparatus 500. Since it is necessary to miniaturize and lighten the wearable apparatus 500 worn by the user, there are large restrictions on a processing performance of a battery or a processing unit in the apparatus or a storage capacity of data. In contrast, since resource restrictions are relatively small in the server system 600, a process of estimating biometric information based on, for example, body motion information can be performed rapidly or a larger amount of data (biometric history information, body motion history information, and the like) can be retained.

Since the information processing system 100 may be able to acquire various kinds of information collected by the wearable apparatus 500, the information processing system 100 is not limited to be connected directly to the wearable apparatus 500. For example, as illustrated in FIG. 24, the wearable apparatus 500 may be connected to another processing apparatus 700 and the information processing system 100 may be connected to the processing apparatus 700 via the network NE. In this case, a portable terminal apparatus such as a smartphone used by a user wearing, for example, the wearable apparatus 500 is considered as the processing apparatus 700. The wearable apparatus 500 and the processing apparatus 700 are connected using the same network as the network NE, but a short-range wireless communication or the like can also be used.

The information processing system 100 according to the embodiment may be realized by the processing apparatus 700 (an electronic apparatus and a portable terminal apparatus in a narrow sense) such as a smartphone rather than the server system 600. A configuration example of this case is illustrated in FIG. 25. Many portable terminal apparatuses such as smartphones have restrictions in a processing performance, a storage region, and a battery capacity compared to the server system 600. However, in consideration of a recent performance improvement, a sufficient processing performance or the like can also be considered to be ensured. Accordingly, when requirements of a processing performance or the like is satisfied, a smartphone or the like can used as the information processing system 100 according to the embodiment, as illustrated in FIG. 25.

In addition, when an improvement in a terminal performance, a use form, or the like is considered, an embodiment in which the wearable apparatus 500 (electronic apparatus) includes the information processing system 100 according to the embodiment, as described above, is not denied either. In this case, the information acquisition unit 110 acquires information from the biometric sensor 200 and the body motion sensor 300 in the same apparatus. In a case in which the information processing system 100 is mounted on the wearable apparatus 500, a request for analyzing or storing data setting a large number of users as a target is low in the information processing system 100 and one user or a few users using the wearable apparatuses 500 may be set as a target. That is, in the processing performance or the like of the wearable apparatus 500, a possibility of a request from a user being satisfied is considered to be sufficient.

That is, the technique according to the embodiment can be applied to a terminal apparatus (a biometric information processing apparatus, a biometric information analysis apparatus, a biometric information measurement apparatus, or a biometric information detection apparatus) including an information acquisition unit that acquires body motion information regarding a subject and a processing unit that estimates biometric information based on the acquired body motion information.

As described above, the information processing system 100 is realized by any one of the server system 600, the processing apparatus 700, and the wearable apparatus 500, but the invention is not limited thereto. For example, a body motion information acquisition process, a biometric information estimation process, and a stamina information estimation process may be realized by a distribution process of a plurality of apparatuses. Specifically, the information processing system 100 may be realized by at least two apparatuses of the server system 600, the processing apparatus 700, and the wearable apparatus 500. Alternatively, another apparatus may perform some of the processes of the information processing system 100 or the information processing system 100 according to the embodiment can be realized by various apparatuses (or a combination of apparatuses).

Some or most of the processes of the information processing system, the electronic apparatus, and the like according to the embodiment may be realized by a program. In this case, when a processor such as a CPU executes the program, the information processing system, the electronic apparatus, and the like according to the embodiment are realized. Specifically, a program stored in a non-transitory information storage device is read and the read program is executed by a processor such as a CPU. Here, the information storage device (a device which can be read by a computer) stores a program, data, and the like and its function can be realized by an optical disc (a DVD, a CD, or the like), a hard disk drive (HDD), or a memory (a card type memory, a ROM, or the like). Then, a processor such as a CPU performs various processes according to the embodiment based on a program (data) stored in an information storage device. That is, the information storage device stores a program (a program causing a computer to execute a process of each unit) causing a computer (an apparatus including a manipulation unit, a processing unit, a storage unit, and an output unit) to function as each unit according to the embodiment.

Thus, the processes according to the embodiment can be realized by a program. The program may be, for example, a program which is read to a processing unit (for example, a DSP) of a device such as a smartphone to be executed.

The embodiment has been described in detail above, but those skilled in the art can easily understand that the embodiment can be modified in many forms without actually departing from the new factors and advantages of the invention. Accordingly, the modification examples are all included in the scope of the invention. For example, throughout the specification or the drawings, terms described along with other broader or equivalent terms at least once can be replaced with other terms in any points of the specification and the drawings. The configurations and operations of the biometric information processing apparatus and the program are not limited to those described in the embodiment, but various modifications can be made.

The entire disclosure of Japanese Patent Application No. 2016-058029, filed Mar. 23, 2016 is expressly incorporated by reference herein.

What is claimed is:

1. An information processing device comprising:
an information acquisition unit that acquires biometric information from a biometric sensor;
a processing unit that generates first report information for reporting stamina information indicating stamina of a user and second report information different from the first report information based on the biometric information; and
an output unit that outputs the first report information and the second report information,
wherein the second report information includes at least one piece of information among second stamina information which indicates the stamina of the user and is different from the stamina information, third stamina information regarding another user, advice information or alert information regarding an activity of the user, and exercise continuable information regarding the user.

2. The information processing device according to claim 1,
wherein in a case in which the information acquisition unit is not able to acquire the biometric information from the biometric sensor, the processing unit performs an estimation process of estimating the biometric information and estimates the stamina information based on coefficient information used for the estimation process.

3. The information processing device according to claim 1,
wherein the processing unit performs an estimation process of estimating the biometric information based on an activity time corresponding to an activity strength of the user and coefficient information corresponding to the activity time and estimates the stamina information based on the coefficient information used for the estimation process.

4. The information processing device according to claim 1,
wherein the information acquisition unit acquires post-activity biometric information from the biometric sensor after the activity of the user stops, and
wherein the processing unit estimates the stamina information based on the post-activity biometric information.

5. The information processing device according to claim 1,
wherein the processing unit estimates the stamina information based on a change in the biometric information by an activity continued by the user.

6. The information processing device according to claim 1,
wherein the processing unit generates stamina information in an uphill exercise or stamina information in a downhill exercise as the second stamina information.

7. The information processing device according to claim 1,
wherein the processing unit generates information regarding an animation in which a character is used, as the first report information.

8. The information processing device according to claim 1,
wherein the processing unit receives stamina correction information for correcting the stamina information from the user and corrects the stamina information based on the stamina correction information.

9. An information processing method comprising:
acquiring biometric information from a biometric sensor;
generating report information for reporting stamina information indicating stamina of a user as first report information based on the biometric information;
generating at least one piece of report information among second stamina information which indicates the stamina of the user and is different from the stamina information, third stamina information regarding another user, advice information or alert information regarding an activity of the user, and exercise continuable information regarding the user, as the second report information; and
outputting the first report information and the second report information.

10. The information processing method according to claim 9,
wherein in the generating, an estimation process of estimating the biometric information is performed in a case in which the biometric information is not acquirable from the biometric sensor in the acquiring, and the stamina information is estimated based on coefficient information used for the estimation process.

11. The information processing method according to claim 9,
wherein in the generating, an estimation process of estimating the biometric information is performed based on an activity time corresponding to an activity strength of the user and coefficient information corresponding to the activity time and the stamina information is estimated based on the coefficient information used for the estimation process.

12. The information processing method according to claim 9,
wherein in the acquiring, post-activity biometric information from the biometric sensor is acquired after the activity of the user stops, and
wherein in the generating, the stamina information is estimated based on the post-activity biometric information.

13. The information processing method according to claim 9,
wherein in the generating, the stamina information is estimated based on a change in the biometric information by an activity continued by the user.

14. The information processing method according to claim 9,
wherein in the generating, stamina information in an uphill exercise or stamina information in a downhill exercise is generated as the second stamina information.

15. The information processing method according to claim 9,
wherein in the generating, information regarding an animation in which a character is used is generated as the first report information.

16. The information processing method according to claim 9,
wherein in the generating, stamina correction information for correcting the stamina information is received from the user and the stamina information is corrected based on the stamina correction information.

17. An information processing device,
wherein biometric information is acquired from a biometric sensor,
wherein first report information for reporting stamina information indicating stamina of a user and second report information different from the first report information are generated based on the biometric information,
wherein the first report information and the second report information are output, and
wherein the second report information includes at least one piece of information among second stamina information which indicates the stamina of the user and is different from the stamina information, third stamina information regarding another user, advice information or alert information regarding an activity of the user, and exercise continuable information regarding the user.

18. The information processing device according to claim 17,
wherein in a case in which the biometric information is not acquirable from the biometric sensor, an estimation process of estimating the biometric information is performed and the stamina information is estimated based on coefficient information used for the estimation process.

19. The information processing device according to claim 17,
wherein an estimation process of estimating the biometric information is performed based on an activity time corresponding to an activity strength of the user and coefficient information corresponding to the activity time and estimates the stamina information based on the coefficient information used for the estimation process.

20. The information processing device according to claim 17,
wherein post-activity biometric information is acquired from the biometric sensor after the activity of the user stops, and
wherein the stamina information is estimated based on the post-activity biometric information.

21. The information processing device according to claim 17,
wherein the stamina information is estimated based on a change in the biometric information by an activity continued by the user.

22. The information processing device according to claim 17,
wherein stamina information in an uphill exercise or stamina information in a downhill exercise is generated as the second stamina information.

23. The information processing device according to claim 17,
wherein information regarding an animation in which a character is used is generated as the first report information.

24. The information processing device according to claim 17,
wherein stamina correction information for correcting the stamina information is received from the user and the stamina information is corrected based on the stamina correction information.
